(19) United States
(12) Patent Application Publication
(10) Pub. No.: US 2026/0187803 A1
(43) Pub. Date: Jul. 2, 2026

(54) PLATFORMS, SYSTEMS, AND ASSOCIATED PROCESSES FOR MEASURING LIFESPAN AND MULTIPLE IN VIVO MOLECULAR BIOMARKERS OF AGING

(71) Applicant: ARIZONA BOARD OF REGENTS ON BEHALF OF THE UNIVERSITY OF ARIZONA, Tucson, AZ (US)

(72) Inventors: George SUTPHIN, Tucson, AZ (US); Samuel FREITAS, Tucson, AZ (US)

(21) Appl. No.: 19/130,482

(22) PCT Filed: Nov. 28, 2023
(86) PCT No.: PCT/US2023/081314

§ 371 Date: May 15, 2025

Related U.S. Application Data
(60) Provisional application No. 63/429,015, filed on Nov. 30, 2022.
(60) Provisional application No. 63/448,539, filed on Feb. 27, 2023.
(60) Provisional application No. 63/448,557, filed on Feb. 27, 2023.

(51) Int. Cl. G06T 7/00; G01N 21/64; G01N 33/50; G06T 7/246; G06T 7/73

(52) U.S. Cl. CPC G06T 7/0016; G01N 21/6456; G01N 21/6486; G01N 33/5085; G06T 7/246; G06T 7/73; G06T 2207/10064; G06T 2207/20104; G06T 2207/30004; G06T 2207/30204

(57) ABSTRACT

Integrated platforms, systems, and associated processes for measuring lifespan, body size and shape, activity, pathology, pigmentation/color, and multiple in vivo molecular biomarkers using multiple cameras are described. The integrated platform includes a plate and trays supported on the plate. Each tray can hold animals. The integrated platform also includes an imaging module with a first camera and a second camera. The first camera can capture first images associated with movement of the animals and the second camera can capture second images and third images associated with biomarkers of the animals.

### CROSS-REFERENCE TO RELATED APPLICATIONS

This application claims priority to each of: U.S. Provisional Pat. Appl. No. 63/429,015 filed on Nov. 30, 2022; U.S. Provisional Pat. Appl. No. 63/448,539 filed on Feb. 27, 2023; and U.S. Provisional Pat. Appl. 63/448,557 filed on Feb. 27, 2023. Moreover, each of U.S. Provisional Pat. Appl. Nos. 63/429,015, 63/448,539, and 63/448,557 are incorporated by reference herein in their entirety.

### STATEMENT REGARDING FEDERALLY FUNDED RESEARCH

This invention was made with government support under Grant No. R35 GM133588 awarded by the National Institutes of Health. The government has certain rights in the invention.

### TECHNICAL FIELD

The present invention generally relates to a high-throughput platform, system, and associated processes for measuring lifespan, body size and shape, activity, and multiple in vivo molecular biomarkers using multiple cameras.

### BACKGROUND

Aging can be characterized by functional deterioration across tissues driven by complex interactions between genetic, environmental, and stochastic processes. Whole-animal mammalian models (e.g., mice) can allow in-depth molecular and physiological characterization of individual animals across life. Tools can be used for high-throughput lifespan measurement in short-lived invertebrate models (e.g., such as worms including for example C. elegans). Some aging studies that use invertebrate models generate population-level data on one or a few physiological parameters. This data can allow primary screening for lifespan or other characteristics.

### SUMMARY

The inventors recognized that an understanding of how interactions between genetic, environmental, and stochastic processes lead to specific physiological outcomes is limited by a lack of robust tools to collect in vivo molecular data about multiple aging mechanisms in parallel. The inventors also recognized that aging studies using whole-animal mammalian models can be prohibitively expensive and can be too time consuming even for moderate-throughput applications. Moreover, the inventors recognized that current aging studies that use invertebrate models and that generate population-level data on one or a few physiological parameters can preclude observation of dynamic molecular interactions and correlations between early-life molecular biomarkers (e.g., expression a gene, or presence of a specific molecule) and late-life outcomes (e.g., longevity, healthspan, activity) in individual invertebrate animals. The inventors also recognized that an impediment to both research designed to understand the molecular biology of aging (e.g., the identification of genes and molecular pathways that drive aging) and anti-aging drug discovery research is a lack of true high-throughput systems for measuring late life health and longevity, and the ability to connect these traits to underlying molecular processes that drive observed changes in healthy aging. Technical capability to simultaneously monitor activity of multiple molecular processes in vivo—and link observed interactions to relevant physiological outcomes like longevity and healthspan (the portion of life spent in good health)—is currently limited, particularly at a scale that allows even moderate throughput screening of candidate interventions.

These limitations and impediments are mitigated or overcome, to a great extent, by an integrated platform according to some aspects of the invention. The integrated platform includes a plate with trays supported on the plate. Each tray is configured to hold animals. The integrated platform also includes an imaging module, which may include a first camera and a second camera. The first camera is configured to capture first images associated with movement of the animals and the second camera is configured to capture second images and third images associated with biomarkers of the animals.

Implementations of the integrated platform may include one or more of the following features. The first images are darkfield images. The imaging module may include a light source for darkfield illumination of a region of interest of the first camera. The light source is a red LED. The second images are brightfield images. The imaging module may include a light source for brightfield illumination of a region of interest of the second camera. The light source is an LED with a triple bandpass filter. The light source is an LED with a triple bandpass filter for fluorescence illumination of the region of interest. The light source is configured to stimulate movement of the animals. The light source is a blue LED. The containers are each configured to contain one of the trays. The third images are fluorescent images. The imaging module may include a light source for fluorescence illumination of a region of interest of the second camera. At least one of the plate or the imaging module is movably mounted to the base. At least one of: the plate is movable relative to the imaging module, or the imaging module is movable relative to the plate. The imaging module is movably mounted to the base and is configured to move in at least one direction relative to the plate. The plate is movably mounted to the base and is configured to move in at least one direction relative to the imaging module. The controller is configured to: coordinate relative positioning of the plate and the imaging module; and control image acquisition from the first camera and the second camera. The housing is configured to control a temperature within an interior of the housing. At least one of the first images, second images, or third images contain data for individual animals, the data being associated with at least one of lifespan of the individual animals, body size of the individual animals, body shape of the individual animals, activity of the individual animals, pathology of the individual animals, pigmentation of the individual animals, color of the individual animals, or in vivo molecular biomarkers of the individual animals. The animals are worms.

Another aspect is directed to a method for image analysis. The method includes inputting, into a computer, first data of animals within trays of an integrated platform. The method also includes extracting, with the computer, second data of the animals from darkfield images collected from a first camera of the integrated platform. The method also includes extracting, with the computer, third data of the animals from brightfield images of the animals collected from a second camera of the integrate platform. The method also includes integrating, with the computer, the first data, the second data, and the third data via a statistical analysis.

Implementations of the method may include one or more of the following features. The method may include: extracting, with the computer, fourth data of the animals from fluorescent images of the animals collected from the first camera; and integrating, with the computer, the fourth data with the first data, the second data, and the third data via the statistical analysis. Extracting the fourth data may include quantifying, with the computer, a fluorescence intensity of biomarkers in each animal. The method may include collecting, with the second camera, the fluorescent images before extracting the fourth data. Collecting the fluorescent images may include collecting at least three fluorescent images of each animal per day. Extracting the second data may include identifying, with the computer, regions of interest or assigning user-defined plate divisions. Extracting the second data may include registering and normalizing, with the computer, the darkfield images. Extracting the second data may include defining, with the computer, movement of animals in the darkfield images. Extracting the second data may include estimating, with the computer, at least one of a lifespan or a healthspan of the animals. Extracting the second data may include estimating, with the computer, the lifespan of the animals from a last day that activity is detected. Extracting the second data may include estimating, with the computer, the healthspan of the animals from a last day that movement of the animals is greater than one body length of the animals. Extracting the third data may include identifying, with the computer, a position of the animals in the brightfield images. Extracting the third data may include quantifying, with the computer, a body parameter of the animals. Collecting the darkfield images may include collecting at least three sets of darkfield images of the animals per day. The method may include stimulating the animals with a light source during the collecting of each set of the darkfield images. Each set of darkfield images may include: a first subset of darkfield images of the animals; a second subset of with at least one darkfield image of the animals taken during light stimulation of the animals and after the first subset of darkfield images; and a third subset of darkfield images of the animals taken after the second subset. The light source is a blue LED. The method may include collecting, with the second camera, the brightfield images before extracting the third data. Collecting the brightfield images may include collecting at least three brightfield images of each animal per day. The animals are worms.

Various additional features and advantages of this invention will become apparent to those of ordinary skill in the art upon review of the following detailed description of the illustrative embodiments taken in conjunction with the accompanying drawings.

### BRIEF DESCRIPTION OF THE DRAWINGS

The following detailed description is better understood when read in conjunction with the appended drawings. For the purposes of illustration, examples are shown in the drawings; however, the subject matter is not limited to the specific elements and instrumentalities disclosed. In the drawings:

FIG. 1 shows a perspective view of an embodiment of an integrated platform;

FIG. 2 shows a perspective view of a first embodiment of a container that can be used with the integrated platform;

FIG. 3 shows a partial cross section view of the first embodiment of the container of FIG. 2;

FIG. 4 shows a perspective view of a second embodiment of a container that can be used with the integrated platform;

FIG. 5 shows a partial cross section view of the second embodiment of the container of FIG. 4;

FIG. 6 shows a perspective view of a third embodiment of a container that can be used with the integrated platform;

FIG. 7 shows a partial cross section view of the third embodiment of the container of FIG. 6;

FIG. 8 shows an example control process for the integrated platform;

FIG. 9 shows an example process for extracting data from darkfield images collected from the integrated platform;

FIG. 10 shows an example process for extracting data from brightfield images and data from fluorescent images collected from the integrated platform;

FIG. 11 shows an example process for analysis of images collected from the integrated platform;

FIG. 12 shows lifespan data of animals;

FIG. 13 shows healthspan data of animals;

FIG. 14 shows age-specific mortality of animals;

FIG. 15 shows animal activity data;

FIG. 16 shows daily activity and lifespan data for animals;

FIG. 17 shows muscle mitochondrial content data for animals;

FIG. 18 shows bacterial infection data for animals;

FIG. 19 shows dual-channel reactive oxygen species data for animals;

FIG. 20 shows stress response data for animals; and

FIG. 21 shows detection and quantification data for animals automatically generated by the integrated platform.

### DETAILED DESCRIPTION

Aspects of this disclosure are directed to integrated platforms, systems, and/or associated processes for simultaneous automated quantification of diverse molecular biomarkers and physiological characteristics of longevity and healthy aging in individual animals such as worms, though the platform is not limited to worms and can be used with cells other animals as well, such as rotifers, other worm and nematode species (e.g., C. briggsae or C. remanei), or fruit fly larva. While the integrated platforms, systems, and/or associated processes of this disclosure can be used for applications in aging science, the integrated platforms, systems, and/or associated processes can additionally or alternatively automate many biological research techniques and will be equally useful across a broad range of biological disciplines including, but not limited to, stress response, toxicology, development, disease specific research, and immunology. The integrated platforms, systems, and/or associated processes according to some aspects of this disclosure can combine automated, high-throughput measurement of longevity and multiple health metrics with simultaneous quantification of in vivo molecular biomarkers in individual worms (e.g., tens of thousands of individual worms in parallel) across lifespan, opening the door to scalable multi-phenotype screening for pharmacological, environmental, nutritional, microbial, and genetic interventions to improve healthy aging and combat age-associated disease.

One aspect of this disclosure is directed to a robotic imaging and analysis platform for automated, high-throughput collection of the central physiological characteristics of interest in aging and other disciplines of biology and health sciences—lifespan, healthspan, body size, body shape, presence of pathology, distance traveled, speed of movement, and daily activity (a metric of health)—in animals such as Caenorhabditis elegans (C. elegans), other worms, rotifers, fruit fly larva, cells, other animals, among other possibilities. The terms “C. elegans” and “worms” as used herein can refer to their respective plain and ordinary meanings, but do not limit the integrated platforms, systems, and associated processes of this disclosure to use with any particular animal including any of the animals described herein. Put differently, any example applications of the integrated platforms, systems, and associated processes of this disclosure that use C. elegans or worms are merely example applications and do not limit the integrated platforms, systems, and associated processes of this disclosure to those particular animals.

The integrated platforms, systems, and/or associated processes of this disclosure can enable time- and resource-efficient multi-phenotype drug and genetic screening, biomarker identification, and molecular interaction studies. The integrated platforms, systems, and/or associated processes of this disclosure can be compatible with a wide range of available models for normal aging, accelerated aging, age-associated disease, toxicology, genetic disease, stress response, immunity, nutrition, development, and many other areas of biology. The integrated platforms, systems, and/or associated processes of this disclosure can also include hardware and software tools (implemented by control systems) for systematic quantification of fluorescent biomarkers, allowing molecular systems to be monitored in living, free crawling C. elegans. The physiological and molecular (i.e., fluorescent) measurements can be measured in the same animals or in distinct populations.

The integrated platforms, systems, and/or associated processes of this disclosure can leverage recent advances in robotics and image analysis to provide automated imaging systems for high-throughput longevity, health, and multichannel fluorescent biomarker quantification in individual worms.

The integrated platforms, systems, and/or associated processes of this disclosure can provide high-throughput systems capable of monitoring diverse in vivo molecular and physiological phenotypes in individual worms across lifespan. The integrated platforms, systems, and/or associated processes of this disclosure can automatically perform robotic imaging and analysis of measurements of lifespan, healthspan, and activity in individual C. elegans. Aspects of this disclosure are also directed to tools for rapid, automated quantification of fluorescent biomarkers across a diverse range of molecular processes. The integrated platforms, systems, and/or associated processes of this disclosure can combine these tools into an integrated robotic imaging platform that can perform autonomous high-throughput quantification of lifespan, healthspan, activity, body size and shape, and multichannel fluorescence in individual worms throughout life.

The integrated platforms, systems, and/or associated processes of this disclosure can simultaneously measure multiple biological mechanisms in vivo. The integrated platforms, systems, and/or associated processes of this disclosure can be highly flexible and can allow researchers to investigate interactions between a broad range of molecular mechanisms. The integrated platforms, systems, and/or associated processes of this disclosure can utilize an extensive panel of multi-biomarker strains across many molecular processes and disease models, which can support a broad spectrum of applications. The integrated platform, related systems, and/or related processes of this disclosure can also be compatible with the extensive set of existing fluorescent biomarker strains currently available, and the potential for future strain construction is virtually unlimited.

The integrated platforms, systems, and/or associated processes of this disclosure go beyond an extension of scale. For example, the integrated platforms, systems, and/or associated processes of this disclosure can enable high content collection of data, allowing many traits to be measured within each animal. This can allow connections to be drawn between different traits by examining both in the same animal. Such connections would not be evident from examining each trait across different sets of animals. Additionally, the integrated platforms, systems, and/or associated processes of this disclosure can monitor the same traits in the same individual animals over time. This can allow dynamic interactions between traits to be observed that might be causally linked, but that occur at different times (e.g., early life changes in the expression in a subset of animals may influence late life health, longevity, or molecular changes in other processes in that same subgroup later in life). Further, the integrated platforms, systems, and/or associated processes can collect data for large numbers of animals in parallel (e.g., thousands of animals per platform, hundreds of thousands to millions of animals across multiple platforms), enabling high-throughput genetic or drug screening for targets that influence any one individual trait, sets of traits of interest, or even the interaction between selected traits. This capability to examine interactions among and across a large set of traits across many individual animals provides value well beyond an equivalent system that would measure the same data independently in distinct populations.

The integrated platforms, systems, and/or associated processes of this disclosure can include hardware and associated control software executed by controllers for collecting imaging data from individually housed animals (such as C. elegans) or cells across lifespan. The integrated platforms, systems, and/or associated processes can be compatible with multiple plate systems for culturing worms either individually or in group populations. A control system (such as a computer that can be separate from the integrated platform) can include, on a non-transitory computer readable medium such as memory, software in accordance with some aspects of this invention. When executed by the control system, the software can collect and process raw images and extract both physiological and fluorescent biomarker data, and software for statistical analysis and presentation of collected data.

As discussed previously, the integrated platforms, systems, and/or associated processes of this disclosure can combine automated quantification of longevity, healthspan, body size, body shape, movement, pathology, and activity analysis of worms with multichannel fluorescence imaging of isolated individual worms in a culture environment designed for long-term maintenance on solid media. The integrated platforms, systems, and/or associated processes can use whole-plate, high-resolution darkfield imaging to monitor movement (e.g., every 8 hours) and infer daily activity, lifespan, and healthspan for each worm. The term “darkfield” as used herein can include the plain and ordinary meaning and/or can include a technique, which can be used in light microscopy, where the specimen can be illuminated with light that has been scattered or refracted, such that only the scattered light enters the objective lens. This can result in a bright image of the specimen against a dark background, enhancing the contrast and visibility of transparent or unstained samples. The integrated platforms, systems, and/or associated processes can use single-worm, high-resolution brightfield microscopy in combination with high-resolution, low-background fluorescence microscopy to quantify body size, shape, and posture, pathology, pigmentation/coloration, and multichannel fluorescence up to multiple times per day for each worm. Both imaging systems can be positioned by a robotic platform allowing parallel monitoring of, for example, 40 plates (96 worms per plate; 3,840 worms in parallel). Data collection and analysis can be completely autonomous once worms are loaded.

The integrated platforms, systems, and/or associated processes of this disclosure can provide several advantages including but not limited to the following. First, the integrated platforms, systems, and/or associated processes of this disclosure can provide autonomous longitudinal multichannel imaging of individual C. elegans, which is compatible with available genetic, environmental, and pharmacological interventions and fluorescent biomarker strains. Second, the integrated platforms, systems, and/or associated processes of this disclosure can utilize validated multi-biomarker transgenic strains that can report activity across a wide range of molecular processes. Third, the integrated platforms, systems, and/or associated processes of this disclosure can utilize validated genetic and transgenic models of age-associated disease with relevant fluorescent biomarkers (e.g., worms transgenically expressing green fluorescent protein (GFP)-tagged amyloid-beta to model Alzheimer's disease). Fourth, the integrated platforms, systems, and/or associated processes of this disclosure can execute software for automated image collection, experiment scheduling, and image processing (i.e., image registration, background normalization and subtraction, error detection, and censoring). Fifth, the integrated platforms, systems, and/or associated processes of this disclosure can execute software for quantification of phenotypes from processed image data for individual worms, including for example: lifespan, healthspan, daily activity, lifetime activity, body size, body shape and posture, presence of pathology (e.g., vulval integrity defects), pigmentation/coloration, quantification of fluorescence intensity across multiple (e.g., 3) fluorescence channels, and identification of fluorescence tissue localization.

The integrated platforms of this disclosure can include a plate. The integrated platforms of this disclosure can include an array of containers for culture trays supported by the plate. The integrated platforms of this disclosure can be sized for standard single-well trays (e.g., OmniTrays), multi-well plates (e.g., 24-well plates), or petri plates (e.g., 35 mm, 60 mm, or 100 mm petri plates). The plates can be arrayed on a 2-dimensional grid in the X-Y plane of the system.

The integrated platforms of this disclosure can include an imaging system, which can capture darkfield images and can stimulate the worms with light (e.g., blue light). A first imaging module (e.g., first camera or plurality of first cameras) can be used to collect whole-plate darkfield images. The resolution of those images can be sufficient to quantify daily worm movement before and after light stimulation, which in turn can be used to estimate lifespan and healthspan. Darkfield images can also be used to quantify worm body size, body, and posture, and pathology. The darkfield imaging module can include a 20 MP low noise monochrome camera for whole plate imaging set in the top of an imaging box with mirrored interior surfaces. The base of the box can be sized to fit around a standard single well tray (e.g., OmniTray), a multi-well plate, or a petri plate. The base of the box is equipped with a series of lights (e.g., red light emitting diodes, LEDs) to provide incident lighting for darkfield illumination of the worms. The box can, in embodiments, be further equipped with other lights (e.g., bright blue (455-465 nm) LEDs) to provide movement stimulus to the worms.

The integrated platforms can also include a second imaging module (e.g., second camera or plurality of second cameras) for brightfield and fluorescence imaging. In embodiments, other types of imaging can be additionally or alternatively performed by the second imaging module including for example scintillation nanoparticle detection; hyperspectral imaging; optogenetics; combinations thereof; among other possibilities. The second camera can be used to capture brightfield and/or fluorescent images of individual worms in each container at higher resolution. These images can be used by the integrated platform to quantify fluorescent biomarkers. Brightfield images can be used to quantify worm body size, body, and posture, pigmentation/coloration, and pathology. This imaging module can include lights (e.g., a white LED brightfield light source, a multichannel (e.g., 3-channel) LED fluorescence excitation light source), a multichannel (e.g., 3-channel) bandpass filter, a camera (e.g., a 5 MP low-noise color camera) to collect images, among other possibilities. In embodiments, the imaging module can include multiple 5MP low-noise monochrome cameras each collecting a different channel (e.g., brightfield; 4′,6-diamidino-2-phenylindole, DAPI; GFP; red fluorescent protein, RFP).

The integrated platforms of this disclosure can include positioning systems for the imaging modules and plates. In embodiments, the plate can remain static, and the imaging modules can be positioned sequentially over each plate for image capture. Alternatively, the plate array can be positioned in the X- and Y-axes under the imaging module, and the imaging module can be positioned in the Z-axis over each plate, among other possibilities. The control system can include, for example, a drive system (e.g., a belt or screw driven X- and Y-axes and screw driven Z-axis driven by four high torque stepper motors, a movable gantry, etc.) that can move the imaging modules and/or the plates. In embodiments, a 3-dimensional rotary design can include disk plates that can be independently positioned circumferentially by, for example, individual high torque stepper motors. The imaging modules can both be positioned axially, for example by lead screw actuators. The darkfield imaging module can be radially fixed, while the brightfield/fluorescence imaging module can be radially positioned by, for example, dual lead screw actuators. Both integrated platforms can provide precise positioning of the darkfield imaging module over each plate, and the brightfield/fluorescent imaging module over each individual well within each plate.

The integrated platforms of this disclosure can include a temperature and light controlled enclosure. Environmental temperature and light are both major determinants of worm lifespan, behavior, and health. The integrated platforms of this disclosure can be housed in a custom incubator to prevent external light exposure and provide an internal light- and temperature-controlled environment. The custom enclosure can monitor temperature in real time and can maintain a constant internal temperature (±1° C.) within the viable temperature range for worms (12 to 26° C.) or be adjusted outside the viable temperature range, either colder (4 to 12° C.) or warmer (16 to 55° C.), for short-term temperature stress experiments using, for example, thermoelectric solid state air conditioning.

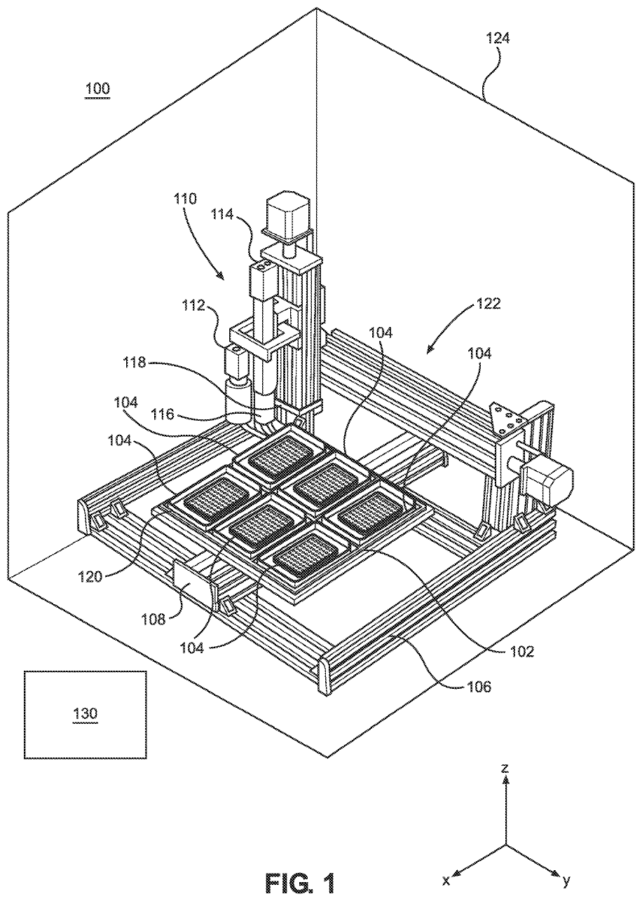

These and other aspects of the integrated platforms, systems, and/or associated processes of this disclosure are shown in FIGS. 1-21 and described further below.

### Integrated Platform

FIG. 1 shows a perspective view of an integrated platform 100 according to some aspects of the invention. The integrated platform 100 can include any of the features of the integrated platforms described previously. For example, the integrated platform 100 can be an autonomous multi-channel imaging platform (e.g., a 2-dimensional grid platform design) for longitudinal tracking individual C. elegans (or cells or other animals such as rotifers, other nematode species, or fruit fly larva, since this disclosure is not limited to C. elegans). The integrated platform 100 can include a plate 102, which can support containers 104 that each can hold C. elegans, cells, or other animals. In embodiments such as shown in FIG. 1, the integrated platform 100 can include one plate 102, which can support six containers 104 and each container 104 can hold ninety-six worms. Though other numbers of plates 102, containers 104, and worms are possible. For example, in embodiments not shown the integrated platform can include multiple plates in, e.g., a three-dimensional, stacked relationship. In additional or alternative embodiments, systems of this disclosure could be scalable to at least eighty-one plates 102 (e.g., with about 19,440 worms total across all plates 102) by increasing the footprint of the integrated platform 100, using different types of plates 102, using multiple integrated platforms 100, among other possibilities.

In embodiments, the plate 102 can be movable in one or more direction relative to a base 106 of the integrated platform 100. For example, the plate 102 can be movably mounted on a track 108 and can be driven linearly along the track using motors, belt or lead screw actuators, combinations thereof, among other possibilities. The integrated platform 100 can include any number of tracks 108 or functional equivalent systems to enable the plate 102 to move in any number of different directions including along any of all of the x, y, and z axes shown in FIG. 1. In alternative embodiments, the plate 102 can be immovably fixed relative to the base 106.

The integrated platform 100 can include an imaging module 110 that can capture images of the worms within the containers 104. In embodiments, the imaging module 110 can include a first camera 112 and a second camera 114, though any number of cameras are possible including only one camera. The first camera 112 can be structured and arranged to capture darkfield images of each plate 102, which in embodiments can be used for quantification of worm lifespan, movement, and activity. In embodiments, the first camera 112 can be a 20 MP CMOS camera. The second camera 114 can, in embodiments, capture 4-channel (e.g., brightfield, DAPI, GFP, RFP) images of individual worms. The images captured by the second camera 114 can be used for quantifying body size, body shape, pathology, and fluorescently labeled biomarker quantification. In embodiments, the second camera 114 can include a 5 MP sCMOS camera mounted to a fluorescence microscope. In embodiments, the second camera 114 can include multiple cameras such as one camera for each of the 4-channels (e.g., brightfield, DAPI, GFP, RFP). In embodiments, the second camera 114 can capture more than 4 channels (e.g., brightfield plus 4 or more fluorescent channels).

The imaging module 110 can be movably mounted to the base 106 above the plate 102. For example, the imaging module 110 can be mounted to a gantry 122 that can move the imaging module 110 along any or each of the x, y, z axes over the plate 102. The gantry 122 and track 108, either alone or in combination, can allow the imaging module 110 and/or plate 102 to be moved such that a field of view of the imaging module 110 can be positioned over any region of interest on the plate 102 including any region of interest within any of the containers 104 provided on the plate 102. In embodiments, the gantry 122 and track 108 can together form a computer numerical control (CNC) platform driven by lead screw actuators that can provide precise, relative x, y, z, positioning of the imaging module 110 and/or plate 102.

The integrated platform 100 can include one more light sources, which can, for example, illuminate the field of view of any or all of the cameras, stimulate movement of the animals, among other possibilities. For example, the integrated platform 100 can include a first light source 116, which can be an LED (or functional equivalent) with a triple bandpass filter for three-channel fluorescence and/or brightfield illumination. The integrated platform 100 can additionally or alternatively include a second light source 118, which can be a blue LED (or functional equivalent) of high intensity that can stimulate animal movement. The integrated platform 100 can additionally or alternatively include a third light source 120, which can be a red LED (or functional equivalent) for darkfield illumination. In embodiments, the first light source 116 and the second light source 118 can be mounted together with the imaging module 110 and can be moved, via the gantry 122, together with the imaging module 110. In embodiments, the third light source 120 can be arranged at the periphery of the plate 102 and can be moved, via the track 108, together with the plate 102. Other mounting arrangements for any or each of the first light source 116, second light source 118, and third light source 120 are possible.

In embodiments, the integrated platform 100 can include a housing 124 that encloses at least the containers 104. The housing 124 can be configured to maintain its interior to set temperatures, such as for example to within +/−1° C. of a target temperature. The target temperature can be within a range of 15° C. to 25° C., though other temperatures also possible. By enclosing at least the containers 104 within the housing 124, a temperature of the animals within the containers 104 can be carefully controlled by an operator. In embodiments, any or all of the plate 102, the base 106, the track 108, the imaging module 110 including one or both of the first camera 112 and the second camera 114, the first light source 116, the second light source 118, and the third light source 120 can be enclosed within the housing 124.

The integrated platform 100 can include a controller 130 (e.g., a computer (with any number of different processors, memories, user interfaces), a control box, combinations thereof, etc.) for controlling any of the systems and processes described herein.

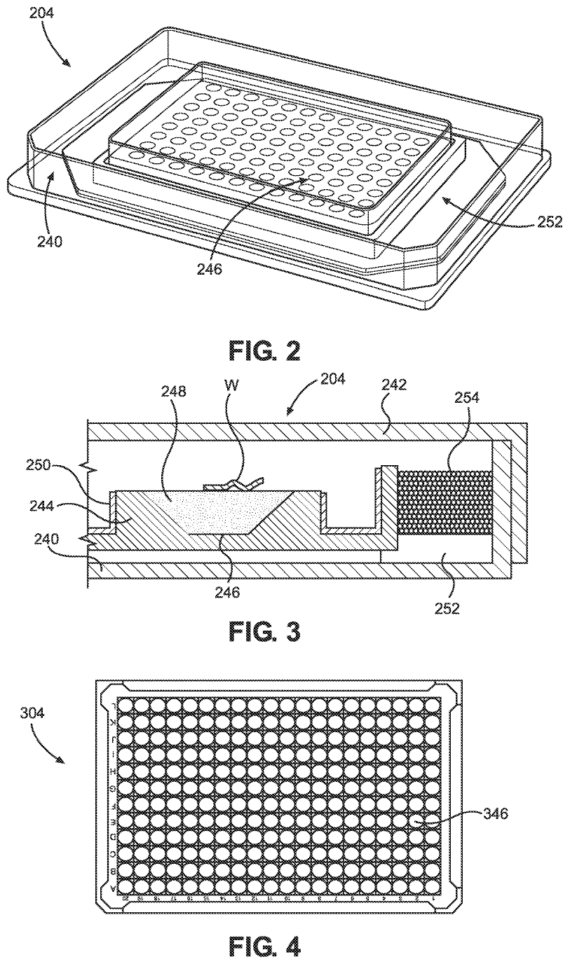

FIG. 2 shows a perspective view of a first embodiment of the container 204 that can be a part of or used with the integrated platform 100. FIG. 3 shows a partial cross section view of the container 204. The container 204 can include any of the features, structures, relationships, etc. described previously with respect to the container 104, and vice versa.

The container 204 can include a base 240 and, in embodiments, a cover 242 that can be removed from the base 240. In embodiments, the cover 242 can be translucent such that the imaging module 110 can view a region of interest within the container 204 through the cover 242. In embodiments, the cover 242 can be formed of a material that can allow oxygen exchange while also preventing or limiting moisture loss, such as a parafilm. The container 204 can include or receive a tray 244 that can be supported by the base 240. In embodiments, the tray 244 can be a microtray (e.g., Terasaki tray), though other trays are possible. The tray 244 can include a number of wells 246 (e.g., 96) that can each receive one or more animals, such as worms W. The wells 246 can be hold a growth media 248. In embodiments, the growth media 248 can include nematode growth media (NGM), S-basal, or other media types either as liquid or solidified with agar, agarose, or low-melt agarose without food or seeded with food (e.g., bacterial or axenic food), though material compositions are possible. In embodiments, the wells 246 can each hold 20 μL (+/−5%) of growth media 248, though other amounts are possible. In embodiments, surfaces of the tray 244 extending between the wells 246 can be coated with an aversive chemical 250 (e.g., palmitic acid, copper sulfate, polyethylene glycol (PEG), among other possibilities), which can discourage the worm from moving out of its respective well 246. The container 204 can include an adapter 252, which can secure the tray 244 within the base 240. In embodiments, the adapter 252 can be 3D printed or molded, though other manufacturing techniques are possible. The container 204 can include water crystals 254 or other functional equivalent to maintain humidity within the container 204. In embodiments, the water crystals 254 can be held in the base 240 around a periphery of the tray 244.

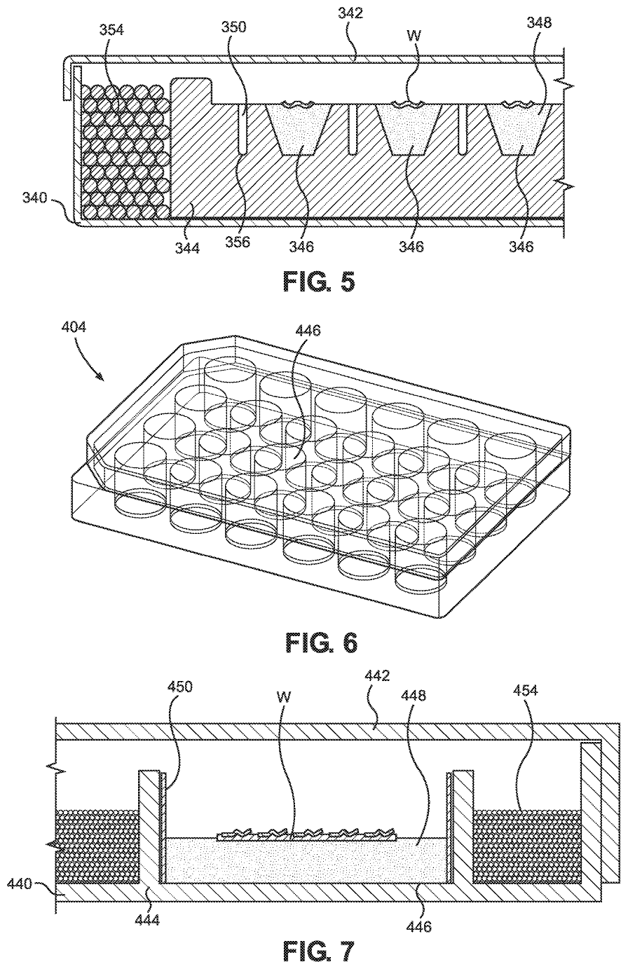

FIG. 4 shows a perspective view of a second embodiment of the container 304 that can be a part of or used with the integrated platform 100. FIG. 5 shows a partial cross section view of the container 304. The container 304 can include any of the features, structures, relationships, etc. described previously with respect to the containers 104, 204, and vice versa. For example, the container 304 can include a base 340, a cover 342, a tray 344, wells 346, growth media 348, an aversive chemical 350, an adapter (not shown), and water crystals 354. In embodiments, the tray 344 can be a custom molded polydimethylsiloxane (PDMS) device with two-hundred and forty wells 346, though other manufacturing techniques, material compositions, number of wells 346 are possible. In embodiments, the wells 346 can be surrounded by moats 356 that can contain the aversive chemical 350.

FIG. 6 shows a perspective view of a third embodiment of the container 304 that can be a part of or used with the integrated platform 100. FIG. 7 shows a partial cross section view of the container 404. The container 404 can include any of the features, structures, relationships, etc. described previously with respect to the containers 104, 204, 304 and vice versa. For example, the container 404 can include a base 440, a cover 442, a tray 444, wells 446, growth media 448, an aversive chemical 450, an adapter (not shown), and water crystals 454. In embodiments, each of the wells 446 can be of a sufficient size to culture populations (e.g., 20-50 worms) within each well 446.

The integrated platform 100 is not limited to use with any of the containers 104, 204, 304, or 404 and can be compatible with other culture systems. For example, the integrated platform 100 can be used with 35 mm, 60 mm, 100 mm or larger petri plates, among other possibilities.

### Control of Image Acquisition Control and of the Integrated Platforms

In embodiments, the positioning motors (e.g., of the gantry 122 and/or track 108) can be controlled by the controller 130. For example, the controller 130 can include GRBL (an open-source machine code standard) or other open-source multi-axis stepper control systems. An example of which is the Openbuilds' Blackbox CNC system running GRBL. The controller 130 can toggle any of the previously described light sources using any solid-state relay control with a toggling signal from either a digital or analog signal. This toggling signal can be generated from any sort of general-purpose input/output (GPIO) type signal from an Arduino, Raspberry Pi, LabJack, or similar systems.

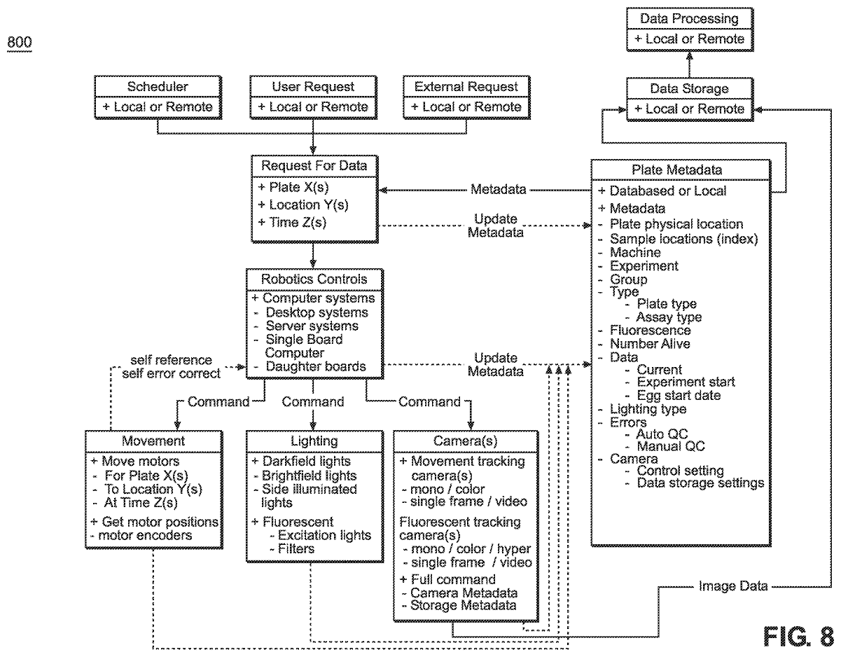

The controller 130 can include custom data acquisition software (stored on non-transitory computer readable media such as memory and executed by one or more control system such as a remote computer). The software, when executed by the control system, can coordinate plate and imaging module positioning, LED activation timing, and image acquisition. FIG. 8 shows a flow diagram for an example control process 800 that can be used to control the operation of the integrated platform 100. However, other control processes can be used to control operation of the integrated platform 100.

Users can define experiment scheduling and experiment-specific timing of image acquisition for both darkfield and brightfield/fluorescent image modules. These parameters can be adjusted for each experiment. In embodiments, the execution of the software by the controller 130 can collect three sets of darkfield images of the animals and one set of fluorescent images of the animals each day. Each darkfield imaging set can include a first subset of darkfield images (e.g., 12 images, though other amounts of images are possible); a second subset (e.g., at least one darkfield image, though other amounts of images are possible) during light stimulation (e.g., blue light stimulation), and a third subset of darkfield images (e.g., 12 images, though other amounts of images are possible) following light stimulation (e.g., blue light stimulation). This schedule can provide accurate and consistent estimates of daily unstimulated activity, stimulated activity, healthspan, and lifespan. This schedule can be adjusted for each experiment.

Each brightfield/fluorescence imaging set can include 3-5 replicate images of each worm in each channel (e.g., brightfield, DAPI, GFP, RFP). Because the worms are free-crawling, collecting multiple images can provide more accurate quantification of whole worm fluorescence intensity, accounting for a subset of images with blur resulting from sudden worm movement.

Image acquisition, as implemented by the controller 130, can include a user alert function that can automatically alert (e.g., via email) designated users in the event of a system error or loss of power, and the capacity to automatically restart following loss of power once power is restored, thus minimizing data loss. In embodiments, the user alert function can automatically alert designated users when an experiment has completed. Completion can be defined by a user as, for example, a specified time period, a number of images, or achievement of experimental goals based on processed data (e.g., no worms remain alive in the experiment).

In embodiments, software, when implemented by the controller 130, can process collected darkfield images. Once images are collected, custom imaging processing software (called “Worm Paparazzi,” described further later), when executed by the controller 130, can carry out a series of image and data processing steps to extract activity, lifespan, body size, body shape, body posture, pathology, and healthspan data from darkfield image sets for each plate. Users can define the experiment name and the experimental conditions contained within each well in the plate. Users can also identify wells that should be excluded from analysis for a variety of reasons (contamination, broken or missing media, worm never loaded, etc.).

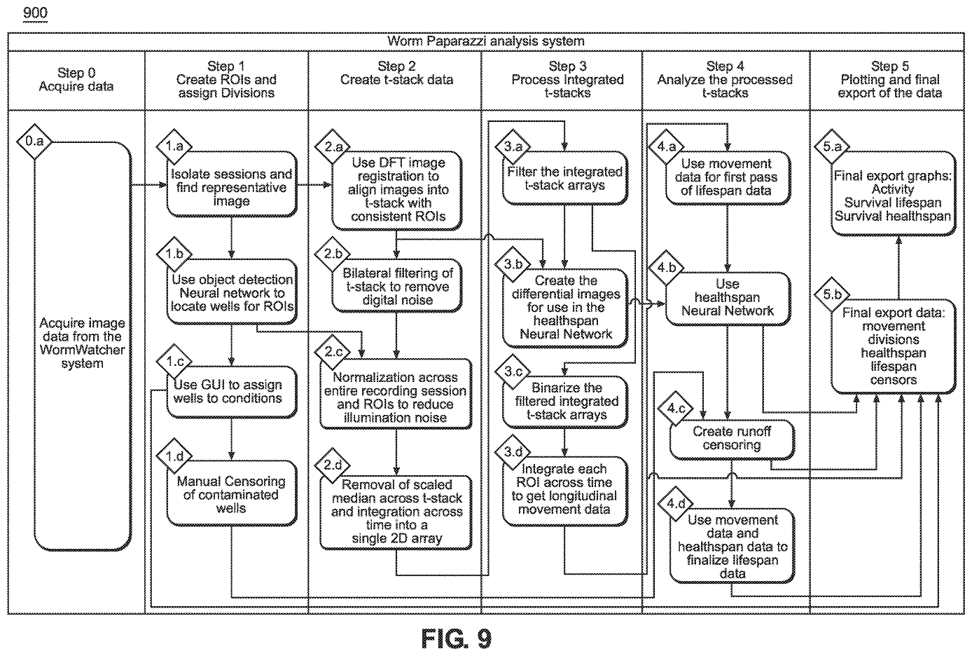

FIG. 9 shows an example flow diagram representing aspects of a Worm Paparazzi process 900 of this disclosure. The Worm Paparazzi process 900, when implemented by the controller 130, can carry out a number of tasks described below and/or shown in FIG. 9.

The Worm Paparazzi process 900 can identify regions of interest (ROIs) and assign user-defined plate divisions (e.g., to define positions in a plate corresponding to different experimental conditions). To identify the position of each worm, a custom neural network (for example custom YOLO (darknet) or DETR (resnet) based networks) can define the area of each whole-plate darkfield image corresponding to each well, and assign the wall to the experimental condition identified by the user.

The Worm Paparazzi process 900 can perform image registration and normalization. Once well positions are identified, images within each series can be registered and normalized. In embodiments, the Worm Paparazzi process 900 can normalize and register the images using, for example, Discrete Fast Fourier Transforms (DFFT/DFT), sped up robust features (SURF) matching to rigidly or affinely align well positions across image sets, among other possibilities. Pixel intensity can then be normalized across image sets and across wells within each image. This can account for differential brightness for wells at different locations on the plate and temporal changes in light exposure for a given well over the course of an experiment. In embodiments, multiple noise-reduction algorithms such as bilinear and Sobel noise filtering, tophat-bottomhat and cross-image histogram illumination normalizations can be employed by the Worm Paparazzi process 900 to remove differences resulting from slight differences in light intensity, which can result from small differences in camera and light source positioning, bacterial growth within the well, noise from light scattering off of objects, and random digital noise in the well such as well edges.

The Worm Paparazzi process 900 can define worm movement in terms of activity, distance traveled, and travel speed. To define worm movement within each well, pixel differences between temporally adjacent images can be calculated within each ROI. Following noise reduction, image-to-image pixel differences can be integrated across sessions to generate unstimulated and stimulated activity for each worm at each session. To define distance traveled and travel speed, the distance between ROI centroids in temporally adjacent images can be calculated and summed across image series (distance traveled), or divided by time between images and averaged across time series (travel speed).

The Worm Paparazzi process 900 can estimate lifespan and healthspan from the activity data generated at each session. Lifespan can be defined as the day after the last day that activity was detected. Healthspan can be defined as the last day that detected worm movement was greater than one worm body length, though other definitions are also possible.

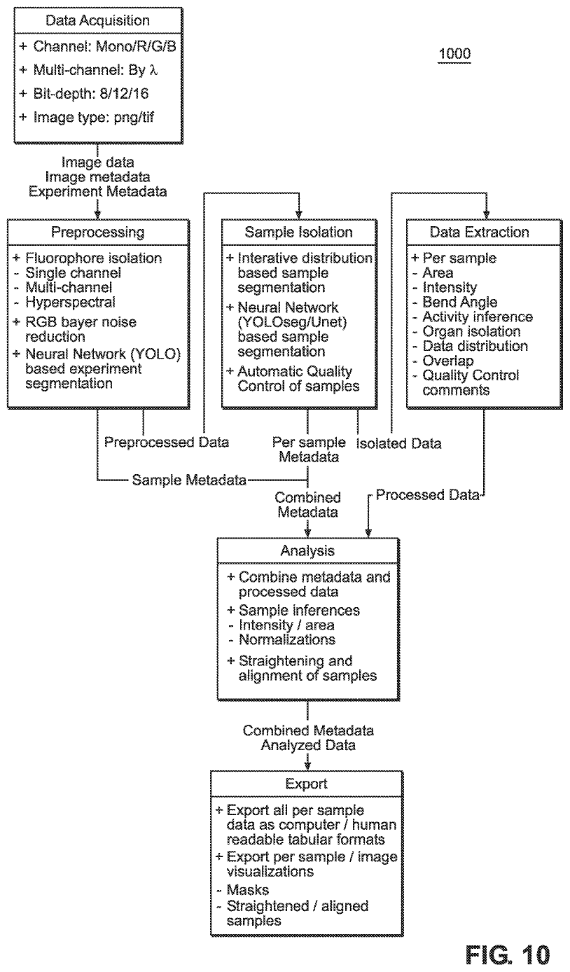

FIG. 10 shows an example flow diagram representing aspects of a LightSaver process 1000. The LightSaver process 1000 can identify worm position in brightfield images. The LightSaver process 1000 can use custom neural networks to (e.g., Unet, YOLO, or YOLOseg based custom networks) precisely define the area of the image representing the worm.

In embodiments, the LightSaver process 1000 can cause the controller 130 to process collected brightfield and fluorescent images. Independently and/or parallel to the Worm Paparazzi process 900, the LightSaver process 1000 can process the brightfield and fluorescence images to extract both body size, shape, and posture data and quantitative fluorescence intensity data. The LightSaver process 1000 can carry out a number of tasks described below and/or shown in FIG. 10.

The LightSaver process 1000 can quantify worm body parameters. The ROI identified in FIG. 9 can be used to fit a standardized geometric model of worm size and shape. This model can be used to define key shape parameters like body area, body length, and body width. Worms crawl with a characteristic sinusoidal motion. Geometric modeling can be used to further estimate established parameters describing worm crawling posture, such as body bend angle. Finally, object recognition convolutional neural networks (e.g., Unet, YOLO, or YOLOseg based custom networks) can be trained on pilot data to identify well-established and visually identifiable forms of pathology, such as vulval integrity defects (Vid).

The LightSaver process 1000 can capture qualitative and quantitative information about worm pigmentation/coloration. Color brightfield images can be collected on the color camera. Color images of sets of worms can be displayed to represent qualitative differences in color intensity or localization (e.g., to intestinal tissue) between animal subsets (e.g., between treatment groups within an experiment). Using color pixel data, the area of a specific color or range of colors can be quantified, and compared to specified area (e.g., the area of the worm or the area of a tissue). Similarly, the intensity of a specific color or color range can be integrated across a specified area (e.g., the area of the worm or the area of a tissue).

The LightSaver process 1000 can quantify fluorescence intensity. Each fluorescence channel can be collected on one channel of the color camera or in separate monochrome cameras. First, background correction can be applied (e.g., color/wavelength-based normalization and isolation from filter/camera/fluorophore/media datasheets) based on the background intensity of the region of the well surrounding each worm. Next, pixel intensity for each channel can be integrated across the area of each worm to generate both a total and area normalized fluorescence intensity for each fluorescent biomarker in each worm at each time point.

Analysis and reporting software can be used for high throughput experiments using worms, such those performed using the integrated platforms. The analysis and reporting software, when executed by the controller 130, can integrate data from user input, the Worm Paparazzi process 900, and the LightSaver process 100, conduct statistical analyses between groups, and generate reports for users. The specific application can be adaptable to different experiments and user-defined outputs to accommodate different experimental goals.

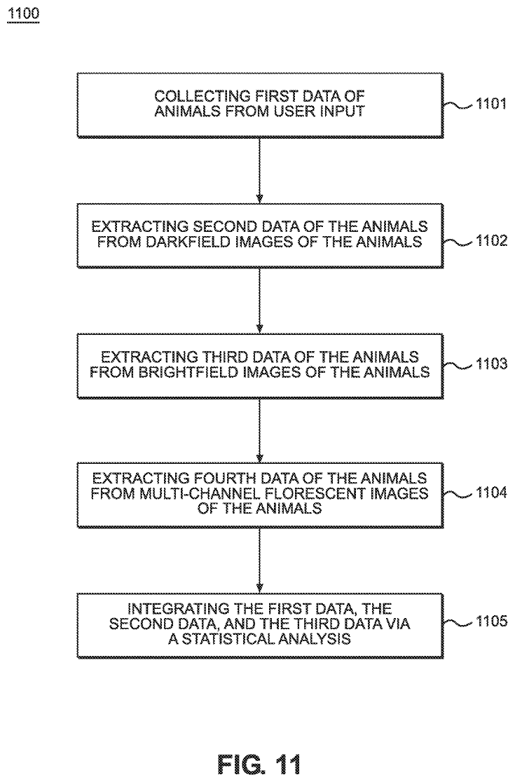

In embodiments, the analysis and reporting software can include a process 1100 for image analysis. FIG. 11 shows an example of process 1100. The process 1100 can be executed using the integrated platforms of this disclosure or independently from the integrated platforms of this disclosure and can be used with other high-throughput experiments.

The process 1100 can include, at step 1101, collecting, with a computer, first data of animals (e.g., worms) from user input, as previously described. First data can include any form of critical or observational data provided by a user about the animals, experimental conditions, or physical location that is not autonomously collected by the imaging system. For example, first data can include animal information such as species; strain name and/or background; genotype; location on experimental plate; information on the state of each animal prior to the start of experiment (e.g., infected vs. non-infected, animal size, or quantification of biomarkers of interest); combinations thereof, among other possibilities. First data can include food source information such as type (e.g., bacteria vs. axenic); composition (for axenic food); strain name and/or background (e.g., for bacterial food); genotype (e.g., for bacterial food); plasmids present (e.g., for bacterial food); worm gene targeted with RNAi plasmid (e.g., for RNAi feeding experiments); concentration; preparation (e.g., growth conditions, heat or UV treatment, sterilization); combinations thereof, among other possibilities. First data can include culture media information such as media type (e.g., nematode growth media, NGM); composition; preparation; name and concentration of added drugs or other compounds not explicit in media composition; location or method used to apply added drugs or other compounds; combinations thereof, among other possibilities. First data can include environmental information such as temperature; timing, nature, and/or concentration/duration/degree of experimental treatments; location of each experimental treatment group on experiment plates; presence and location of contamination; action taken to mitigate contamination (e.g., animal moved or censored); light level, type, and/or duration; information on environmental stimulus (e.g., plate vibration, exposure to bright light); oxygen level; humidity; combinations thereof, among other possibilities. First data can include other information such as statistical methodology (e.g., details of power analysis used to design experiment); censoring method used; combinations thereof; among other possibilities.

The process 1100 can include, at step 1102, extracting, automatically with the computer, second data of the animals from darkfield images. Second data can be data collected by the darkfield imaging module, as previously described. The second data can be used to measure many characteristics of the animals, as previously described. Second data and derivative characteristics can include for example raw data such as static images of animals on experimental media; static image series (aka video); image meta data (time/date, exposure time, size, resolution, location of image in series, etc.); plate/experiment/location identity, combinations thereof, among other possibilities. Second data can include derivative characteristics quantified from raw data such as body area; body length; body width; body bend angle; body shape; distance traveled; travel speed; activity level before stimulus; activity level after stimulus; animal position in well or on plate; combinations thereof, among other possibilities.

In embodiments, extracting the second data from the darkfield images at step 1102 can include any of the features of the previously described Worm Paparazzi process 900. For example, in embodiments extracting the second data comprises identifying regions of interest or assigning user-defined plate divisions. In embodiments, extracting the second data comprises registering and normalizing the darkfield images. In embodiments, extracting the second data comprises defining movement of animals in the darkfield images. In embodiments, extracting the second data comprises estimating a lifespan or a healthspan of the animals.

The process 1100 can include, at step 1103, extracting, automatically with the computer, third data of the animals from brightfield images of the animals. Third data can include data collected by the brightfield imaging module, as previously described. The third data can be used to measure many characteristics of the animals, as previously described. The third data can include raw data such as for example static images of animals on experimental media; image meta data (time/date, exposure time, size, resolution, etc.);
plate/experiment/location identity; combinations thereof; among other possibilities. The third data can include for example derivative characteristics quantified from raw data such as for example; body area; body length; body width; body bend angle; body shape; animal position in well or on plate; identification of presence of pathology (e.g., vulval rupture, bagging); integrity and area of organs (e.g., intestine, germline, pharyngeal pump); color or pigmentation; combinations thereof; among other possibilities. In embodiments, extracting the third data from the brightfield images can include any of the features of the previously described LightSaver process 1000. For example, in embodiments extracting the third data comprises identifying a position of the animals in the brightfield images. In embodiments, extracting the third data comprises quantifying a body parameter of the animals.

The process 1100 can include, at step 1104, extracting, automatically with the computer, fourth data of the animals from fluorescent images of the animals. Fourth data can include data collected by the multi-channel fluorescent imaging module, as previously described. The fourth data can be used to quantify specific details of fluorescently labeled biomarkers. The fourth data can include for example raw data such as static images of animals on experimental media in multiple fluorescent channels; image meta data (time/date, exposure time, size, resolution, location of image in series, etc.); plate/experiment/location identity; combinations thereof; among other possibilities.

The fourth data can include derivative characteristics quantified from raw data such as for example animal position in well or on plate; area of fluorescence in image; location of fluorescence in image; intensity of fluorescence at each pixel; integrated intensity of fluorescence within worm; background fluorescence intensity; background-corrected fluorescence intensity of each pixel in animal; integrated intensity of background-corrected intensity; distribution of intensity across worm area; combinations thereof; among other possibilities. In embodiments, extracting the fourth data from the fluorescent images can include any of the features of the previously described LightSaver process 100. In embodiments, extracting the fourth data can include quantifying a fluorescence intensity and area of biomarkers in each animal.

The process 1100 can include, at step 1105, integrating the first data, the second data, and the third data via a statistical analysis. In embodiments, the step 1105 can include integrating the first data, the second data, the third data, and the fourth data via the statistical analysis. Integrating the data can include generating integrated data derived from combining data from first, second, third, and fourth data. The integrated data can include for example correlation within or between quantified data from each category; regression analysis of one variable as a function of one or more other variables within or across data types, and within or across experimental groups; summary statistics of data within and across categories (mean, median, mode, standard deviation, standard error, etc.), and within or across treatment groups; quantification of fluorescence (or other extracted image data) on a subset of one image as defined by an area identified in another image (e.g., integrated fluorescence intensity within an area of a selected fluorescence image based on a masked area selected from a brightfield image taken of the same animal); statistical comparison (e.g., log rank test, t test, or other appropriate test) between experimental groups; composite images combining data from two or more images within or across data types; data derived from machine learning or AI algorithms applied to one or more images within or across data types; combinations thereof, among other possibilities. The integrated data can be reported to the user.

A non-exhaustive list of analyses that can be aspects of the process 1100 includes time-to-event analysis (aka survival analysis) for lifespan, healthspan, or other user defined event parameters (e.g. onset of a specific pathology), daily activity, healthspan relative to lifespan, body area, body length, body width, body bend angle, fraction of worms presenting a specific pathology, stage of specific pathologies, intensity of specific fluorescent biomarkers, age-specific correlation of two parameters, correlation of age-integrated values from time-specific parameters (e.g. fluorescent intensity of GFP on day 2) with life-time parameters (e.g. lifespan), lifetime trajectories of any parameter measured daily (e.g. body size or fluorescence intensity) for individual worms or selected populations, pixel-by-pixel ratios between co-expressed fluorescent biomarkers, quantification of changes in subcellular localization for fluorescently labeled proteins, combinations thereof; among other possibilities.

In embodiments, the process 1100 can include assessing lifespan and healthspan data from neural network analysis (such as calculated movement across time periods, colocalization of movement profiles, animal shape, size, and color) of collected images of phenotypic results from experimental data points for individual animals isolated in separate wells. In embodiments, the process 1100 can include or be utilized with 1) additional technology for automated image collection, experiment scheduling, and image processing (i.e., image registration, background normalization and subtraction, error detection, and censoring), 2) software for quantification of the following phenotypes beyond lifespan and healthspan from processed image data for individual worms: daily activity, lifetime activity, body size, body shape and posture, presence of pathology (e.g., vulval integrity defects), quantification of fluorescence intensity across one or more fluorescence channels, identification of fluorescence tissue localization, and quantification of ratios between fluorescent channels compatible with available multi-channel biomarkers; and/or 3) a separate pipeline encompassing the analyses outlined in (1) and (2) for animals cultured together in small populations across a range of plate formats (e.g., microtrays, Terasaki trays, WorMotels, multi-well plates, petri plates).

The process 1100 can enable rapid and simultaneous extraction of quantitative data from images taken of worms cultured in isolation or in groups including longevity, healthspan, movement, activity, body size, body shape, body posture, pathology, pigmentation/coloration, and multiple fluorescently tagged molecular biomarkers. This capacity enables rich multifaceted data to be extracted from individual animals that has applications across many disciplines, including aging, immune function, development, stress response, and models of many human diseases. The process 1100 can further enable different data types to be compared and correlated with each other across individual animals within a population.

### Representative Data

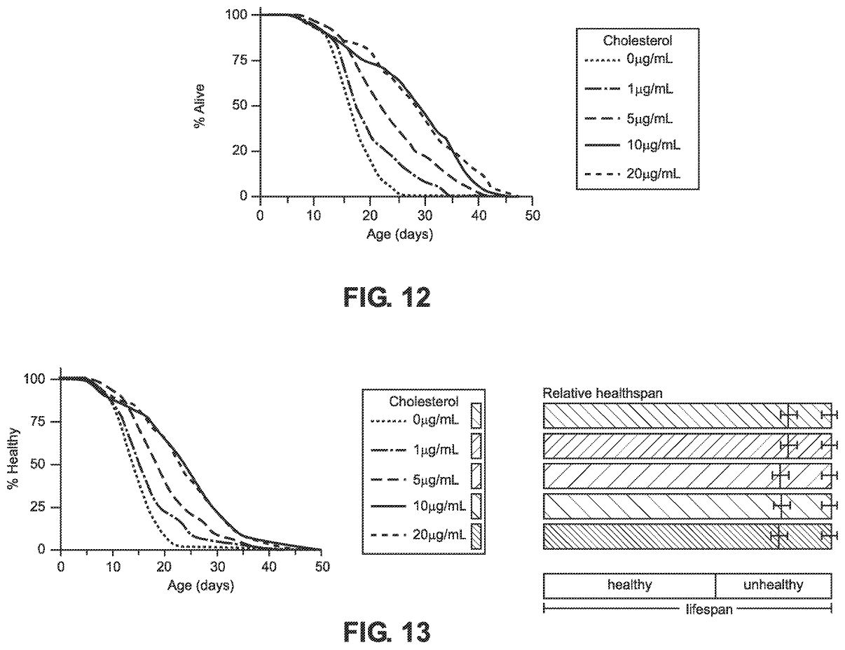

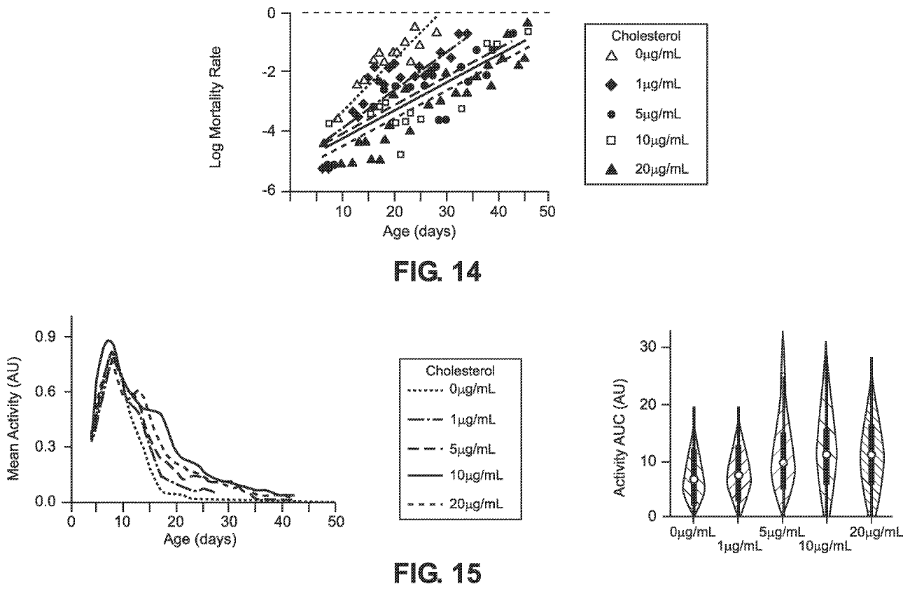

FIGS. 12-21 show representative data generated from integrated platforms, systems, and/or associated processes, as previously described. FIG. 12 shows fully autonomous lifespan data generated from the previously described integrated platform and associated processes. FIG. 13 shows fully autonomous healthspan data generated from the previously described integrated platform and associated processes. FIG. 14 shows fully autonomous age-specific mortality data generated from the previously described integrated platform and associated processes. FIG. 15 shows fully autonomous worm activity (total activity shown; stimulated and unstimulated activity are measured independently) data generated from the previously described integrated platform and associated processes.

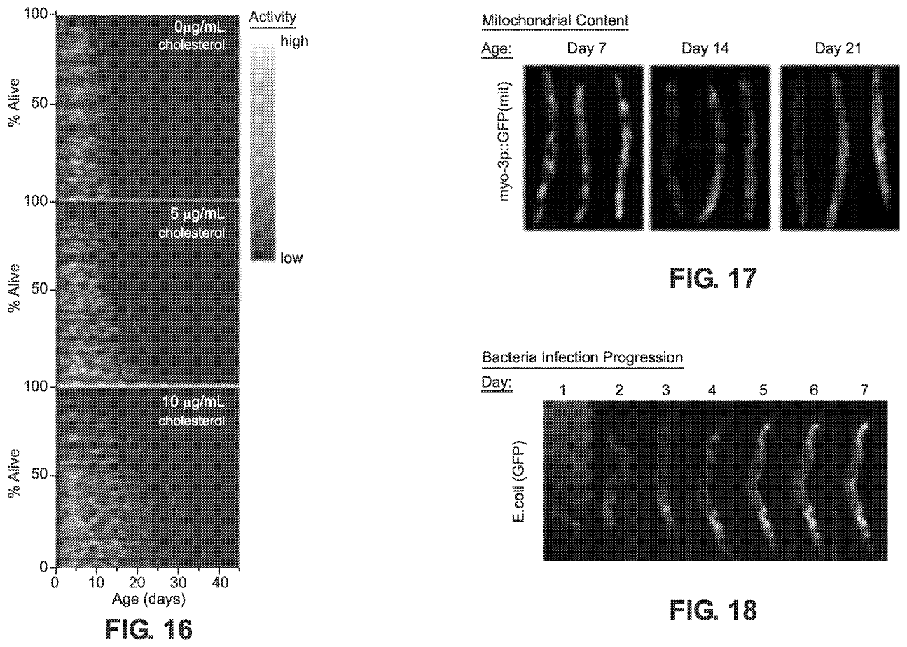

FIG. 16 shows daily activity represented as individual activity traces (heatmap) correlated with lifespan (red points) for each worm. This sample data shows a cholesterol dose-response experiment collected and analyzed autonomously.

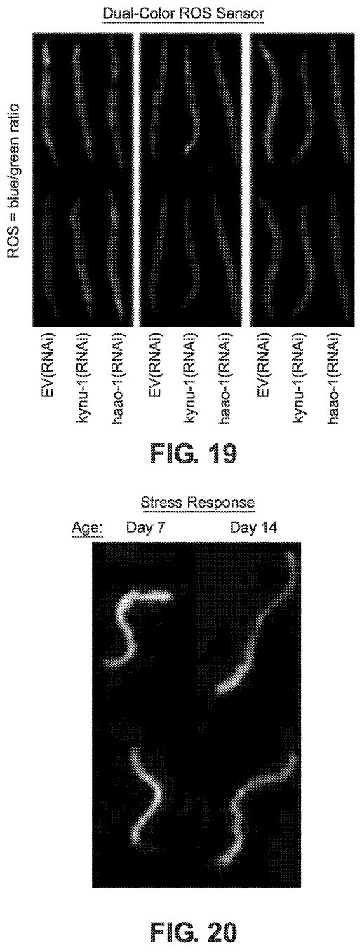

FIGS. 17-20 show a combination of those metrics with periodic measurement (user-defined interval) of three or more fluorescence channels for each worm, which can be used to quantify a range of transgenically expressed biomarkers. Non-limiting examples of biomarkers that can be quantified by the system and methods of this disclosure include muscle mitochondrial content (FIG. 17), progression of GFP-labeled bacterial infection (FIG. 18), dual-channel reactive oxygen species (ROS) detection (FIG. 19), and expression of an extensive panel of stress response reporters (FIG. 20).

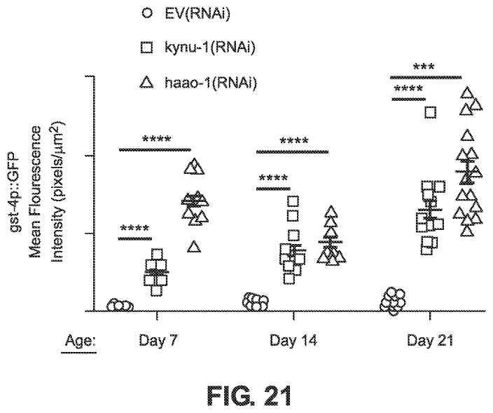

FIG. 21 shows that the integrated platform and associated methods of this disclosure can automatically perform detection and quantification tasks (e.g., sample data shows the response of an oxidative stress response reporter to RNAi knockdown of two genes, kynu-1 and haao-1 over time). Examples of proposed multi-channel fluorescence biomarker strains that can be validated using the integrated platform and associated methods of this disclosure include: multiple stress response pathway reporters; multiple aging pathway transcriptional reporters, dual channel ROS detection; dual channel energy sensors (ATP/ADP ratio, NAD/NADH ratio); multi-isoform reporters for individual genes; infection tracking using labeled bacteria; among other possibilities. Single worm darkfield or brightfield images can also allow quantification of body size (e.g., length, width, area), shape (e.g., aspect ratio), and posture (e.g., bend angle).

It will be appreciated that the foregoing description provides examples of the invention. However, it is contemplated that other implementations of the invention may differ in detail from the foregoing examples. All references to the invention or examples thereof are intended to reference the particular example being discussed at that point and are not intended to imply any limitation as to the scope of the invention more generally. All language of distinction and disparagement with respect to certain features is intended to indicate a lack of preference for those features, but not to exclude such from the scope of the invention entirely unless otherwise indicated.

1.-20. (canceled)

21. A method for image analysis comprising:
inputting, into a computer, first data of animals within trays of an integrated platform;
extracting, with the computer, second data of the animals from darkfield images collected from a first camera of the integrated platform;
extracting, with the computer, third data of the animals from brightfield images of the animals collected from a second camera of the integrated platform; and
integrating, with the computer, the first data, the second data, and the third data via a statistical analysis.

22. The method of claim 21, further comprising:
extracting, with the computer, fourth data of the animals from fluorescent images of the animals collected from the first camera; and
integrating, with the computer, the fourth data with the first data, the second data, and the third data via the statistical analysis.

23. The method of claim 21, wherein extracting the second data comprises identifying, with the computer, regions of interest or assigning user-defined plate divisions.

24. The method of claim 21, wherein extracting the second data comprises registering and normalizing, with the computer, the darkfield images.

25. The method of claim 21, wherein extracting the second data comprises defining, with the computer, movement of animals in the darkfield images.

26. The method of claim 21, wherein extracting the second data comprises estimating, with the computer, at least one of a lifespan or a healthspan of the animals.

27. The method of claim 26, wherein extracting the second data comprises estimating, with the computer, the lifespan of the animals from a last day that activity is detected.

28. The method of claim 26, wherein extracting the second data comprises estimating, with the computer, the healthspan of the animals from a last day that movement of the animals is greater than one body length of the animals.

29. The method of claim 21, wherein extracting the third data comprises identifying, with the computer, a position of the animals in the brightfield images.

30. The method of claim 21, wherein extracting the third data comprises quantifying, with the computer, a body parameter of the animals.

31. The method of claim 22, wherein extracting the fourth data comprises quantifying, with the computer, a fluorescence intensity of biomarkers in each animal.

32. The method of claim 21, further comprising collecting, with the first camera, the darkfield images before extracting the second data, wherein collecting the darkfield images comprises collecting at least three sets of darkfield images of the animals per day.

33. The method of claim 32, further comprising stimulating the animals with a light source during the collecting of each set of the darkfield images.

34. The method of claim 33, wherein each set of darkfield images comprises:
a first subset of darkfield images of the animals;
a second subset of with at least one darkfield image of the animals taken during light stimulation of the animals and after the first subset of darkfield images; and
a third subset of darkfield images of the animals taken after the second subset.

35. The method of claim 34, wherein the light source is a blue LED.

36. The method of claim 21, further comprising collecting, with the second camera, the brightfield images before extracting the third data.

37. The method of claim 36, wherein collecting the brightfield images comprises collecting at least three brightfield images of each animal per day.

38. The method of claim 22, further comprising collecting, with the second camera, the fluorescent images before extracting the fourth data.

39. The method of claim 38, wherein collecting the fluorescent images comprises collecting at least three fluorescent images of each animal per day.

40. The method of claim 21, wherein the animals are worms.
